(19) United States
(12) Patent Application Publication
(10) Pub. No.: US 2002/0107519 A1
(43) Pub. Date: Aug. 8, 2002

(54) Dual spreader flange-tube vertebral stabilizer

(76) Inventors: Robert A. Dixon, Powell, OH (US); Donald J. Hackman, Upper Arlington, OH (US)

Correspondence Address: DONALD J. HACKMAN, 3499 KIRKHAM RD, COLUMBUS, OH, 43221, US

(21) Appl. No.: 10/067,700

(22) Filed: Feb. 5, 2002

Related U.S. Application Data
(60) Provisional application No. 60/266,462, filed on Feb. 5, 2001.

(51) Int. Cl. A61B 17/58

(52) U.S. Cl. 606/61

(57) ABSTRACT

This invention comprises a device and a method of maintaining spinal exposure, distraction, alignment and temporary fixation. Additionally provided for, is tooling guidance for the preparation of an intervertebral space for an implant. The device provides a means for attaching a tube or other attachment to guide tools that will prepare the vertebrae for accepting an implant. At any time during the procedure the attachment may be removed to inspect or remove debris from the disc space and the vertebral end plates without disturbing the distraction and temporary fixation.

### CROSS-REFERENCES TO RELATED APPLICATIONS:

This patent application was preceded by: Provisional Patent#60/266,462 with a file date of Feb.5, 2001

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

Spinal fusions are performed to treat degenerative diseases, deformities, and trauma. These problems generally cause or allow displacement or rotation of a vertebra relative to the adjacent vertebrae. The objective of spinal implants is to facilitate realignment and/or fixation of spinal elements for fusion. It has been demonstrated in clinical studies, that surgeries using spinal implants are more effective in providing structure and rigidity to the spine, than surgeries in which implants are not used. The majority of existing spinal implants use metal rods or plates to restrict the relative motion of the adjacent vertebra while fusing. Once the two vertebrae are fused there is no longer a need for the rods or plates, which may later cause complications. Threaded bone dowels or other prostheses may be implanted for fusion without rods or plates and remain within the vertebrae.

A bone fussion, using a threaded cylindrical implant, may be performed between two adjacent vertebrae to restore the space originally occupied by a disc. To remove the cartilage and to increase the surface area of contact between the flat vertebral end plates and the cylindrical dowel, it is necessary to prepare the flat vertebral end plates to a partial cylindrical concave surface to conform to the dowel.

In the prior art, a distractor consisting of a tube with two protruding tangs was used for initial distracting, maintaining distraction and vertebral position, and acting as a guide during machining and implantation. These tangs are referred to in the literature as extended outer sleeves. These tangs are hammered into the disc space to force and maintain distraction and position of the tube in the intervertebral space. The hammering or impacting may cause trauma to the vertebrae, the ligaments, the blood vessels, and the nerves. Once the tube is in place it cannot be removed until the dowel is implanted. This does not allow the surgeon to inspect the disc space, the thread depth, or the end plate preparation condition. Since the tangs are tapered, any movement of the tube causes a component of the holding force to tend to dislodge the sleeve, thereby allowing movement of the tube and vertebrae with respect to each other. This movement provides for unreliable machining. Additionally, the tangs do not hold the tube reliably and they allow trapezoidal deformation, especially with the cervical vertebrae, which are smaller than the lumbar vertebrae. In other designs wedges, paddles, or plugs distract the vertebra prior to inserting the tangs, however the same tube instabilities and vision obstructions remain.

U.S. Pat. No. 6,080,155 has many similarities to the present patent, however the 155 patent discloses a tube with tangs, which are driven or hammered into the disc space. It does not provide for removing the tube and retaining distraction and it has no clearance undercuts to avoid the vertebral protrusions. These vertebral protrusions are not accurately portrayed in the 155 patent figures.

U.S. Pat. No. 5,899,908 discloses a tube with small teeth, which are driven or hammered into the vertebra. Experience has shown that these teeth will not retain the distraction while the tube is guiding the tools inside of the tube. These teeth also do not provide for removal of the tube for inspection while retaining distraction. The present patent, as described below, provides a more stable and more versatile system that will eliminate these deficiencies.

### BRIEF SUMMARY OF THE INVENTION

The purpose of the present invention is to provide a device and a method to prepare the flat surfaces on the intervertebral end plates to receive an intervertebral prosthesis implant. This device comprises a flange, two distractors, a guide tube, and attachment screws. The distractors are positioned between the vertebral end plates and produces spreading or distraction of the vertebra. This distraction will assure that the disc height is restored at the time of the operation. Restoring disc height provides for opening of the nerve foramen and for ligamentotaxis. The need and purpose of these are well known to those practiced in the art. The disc need not be removed before distraction. The distractor stems may also act as soft tissue retractors.

Once the vertebrae are spread, the edges of the flange are placed over the distractor stems. This will insure that the flange tube hole is centered between the vertebral end plates. When the flange is in place, attachment screws may be placed through the holes in the flange tabs and into the adjacent vertebrae to maintain rigid vertebral spacing and orientation while the tooling and implant are in use. The device rigidly fixes the vertebrae to allow accurate tooling by limiting translation/rotational motion as is allowed in prior art. The tube aligned to the flange assures that the subsequent tooling will be centered on and parallel to the vertebral end plates. This in turn can facilitate removal of an appropriate amount of material from each adjacent vertebra. The implant can then be inserted through the flange. After the machining is completed the implant can be guided through the tube and be contained in place with a holding means, preferably a screw thread. Alternately, the tube may be left off and the implant can be placed through the flange orifice. These machining and prosthesis insertion operations are well known to those skilled in the art.

### BRIEF DESCRIPTION OF THE SEVERAL VIEWS OF THE DRAWINGS

The present invention will be better understood from the following detailed description of the preferred embodiment.

FIG. 1 is an isometric view of the vertebral distraction stabilizer showing the flange, the attachment screws, the distractors, and the removable tube.

FIG. 2 is an exploded isometric view of the stabilizer system.

FIG. 3a is an isometric view showing the distractors inserted into the disc space.

FIG. 3b is an isometric view showing the distractors, with the inferior vertebra removed to show the distractors.

FIG. 3c is an isometric view showing the distractors rotated to distract the vertebrae.

FIG. 3d is an isometric view showing the distractors rotated with the inferior vertebra removed to show the distractors.

FIG. 4 is a side sectional view, along the line 4-4 of FIG. 3a, of a collapsed disc showing a paddle distractor inserted into the disc space.

FIG. 5 is a side sectional view, along the line 5-5 of FIG. 3c, of a paddle distractor inserted into the collapsed disc and rotated 90 degrees to spread two adjacent vertebrae, restoring the disc height.

FIG. 6 is a side sectional view, along the line 6-6 of FIG. 7, of a vertebral disc space with the distractor and the tube removed and the attached flange maintaining the distraction. The disc space and vertebral end faces are open and distracted to be inspected by the surgeon.

FIG. 7 is an anterior view showing the flange attached, holding the distraction. The disc space, the vertebral end plates, and the vertebral protrusion are visible.

FIG. 8 is a side sectional view, along the line 8-8 of FIG. 1, of the guide tube attached to the flange and the reamer cutting the vertebral end plate.

FIG. 9 is a side view of the thread tap.

FIG. 10 is a side sectional view of the implant, along the line 10-10 of FIG. 1.

### DETAILED DESCRIPTION OF THE INVENTION

In the description upper or superior refers to the vertebra nearer to the patients head and lower or inferior refers to the vertebra nearer the patients feet. It is also understood that “fixed” and “rigid” are relative terms not implying zero measurable motion, but much less motion relative to the adjacent vertebra than before installation of the distraction stabilizer system. For simplification the distraction stabilizer system is described as a lumbar device in one of many conceivable embodiments. That is not to imply that this is the only embodiment within which the distraction stabilizing system can be configured. The components may be fabricated from metal or polymers or other materials.

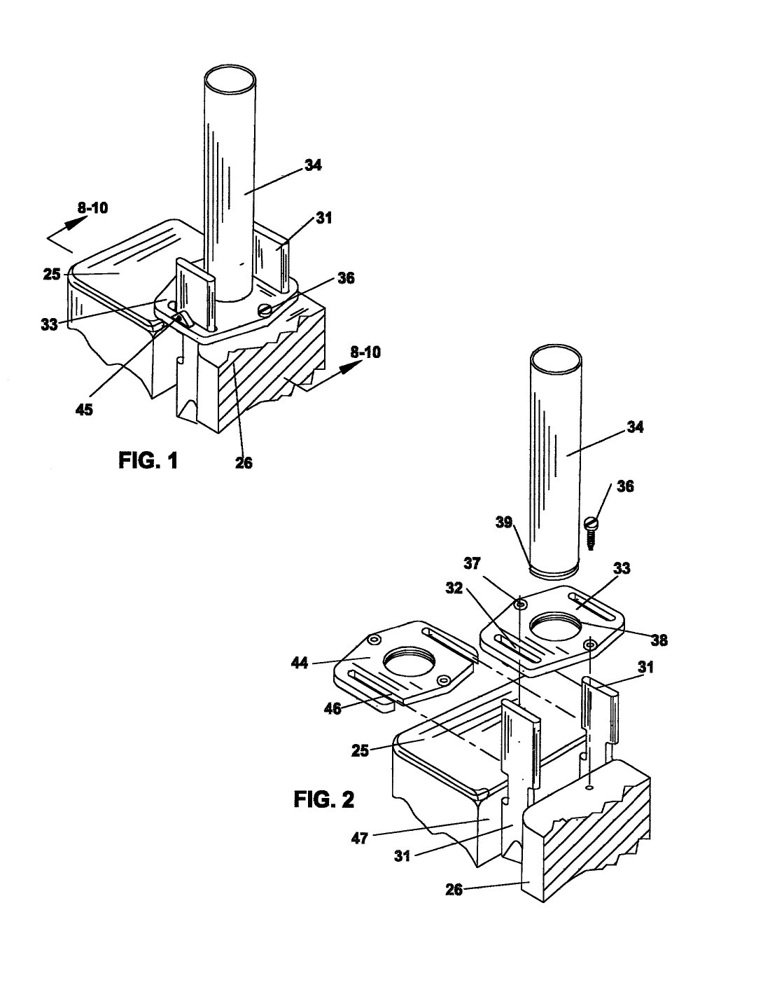

FIG. 1 shows the dual spreader flange-tube vertebral stabilizer maintaining distraction. FIG. 2 shows the individual components. Unlike stabilizing plates or rods, which stress share or stress shield the implant; this dowel implant supports the center of the vertebral end plates and forms a series compressive structure between the two vertebrae. The compressive forces are caused by the gravitational force acting on the upper vertebrae and the tensile forces between the two vertebrae by virtue of ligamentotaxis. This loading maintains compression between the implant and the machined surfaces of the vertebrae. Studies have shown that a device that allows the fusion to remain in compression will tend to lessen joint separation and increase the fusion rate by reducing the stretching rupture and shear of the forming blood vessels and by providing load sharing to the graft material.

The Herniated Disc

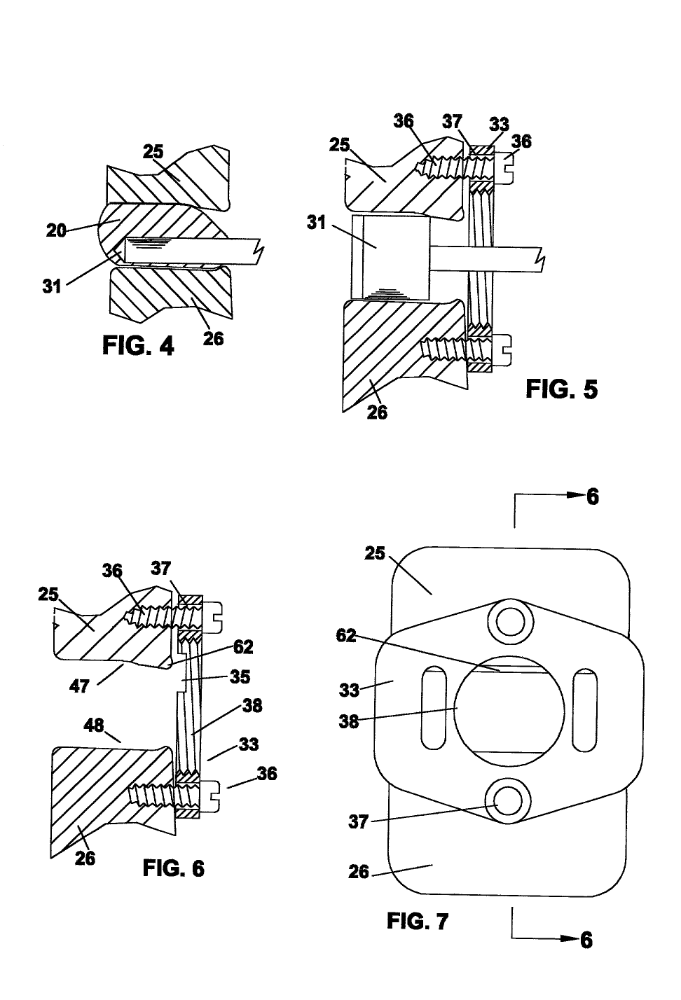

A collapsed disc 20 is shown in FIG. 4. When spinal discs rupture or bulge from injury or from degeneration, the space between two adjacent vertebrae 25 and 26 decreases as shown in FIG. 4. Frequently the bulging does no harm, but if it compresses against the spinal cord or a nerve, it may cause pain, loss of sensation, or weakness. Additionally, the bulge or herniation may indicate a torn or painful disc joint and allowing abnormal motion. In these situations, when surgery is indicated, it is generally safer to replace the disc with a rigid implant and accept the loss of motion that the disc once provided. The discs can be easily removed after distraction, if access is provided to the surgeon. With single piece tang-distractor tube systems, the tube cannot be removed for inspection without releasing the distraction. The tangs also give unreliable stabilization as well as unreliable positioning of the machining surfaces.

The Vertebral Distractor

FIGS. 4 and 5 show paddle type distractors 31. When rotated 90 degrees, as shown in FIG. 5, the disc space height is restored to allow for preparation and implantation between two adjacent vertebrae. The distal end of the distractors may have a knife-edge to aid in removing or separating the disc and facilitating the placement of the distractors. The distractors also act as soft tissue retractors.

The Distraction Stabilizing (Temporary/Fixating) Flange

Once the vertebrae are spread, the flange slots 32 in FIG. 2 are placed over the distractor stems 31 where it is centered upon the disc space to insure that the flange 33 is concentric with the desired dowel location. The attachment screws 36 are placed through the flange tab holes 37 and threaded into the adjacent vertebrae 25 and 26, to maintain the vertebra's relative position while the tooling and implanting operations are taking place. Attaching the flange without a guide tube in place allows the surgeon to have more working space to install the flange attachment screws. The flange may be made of a single, composite, or a plurality of materials. After the flange is attached, the surgeon may remove the distractors or they may be left in place and they may be held with flange clamps 45.

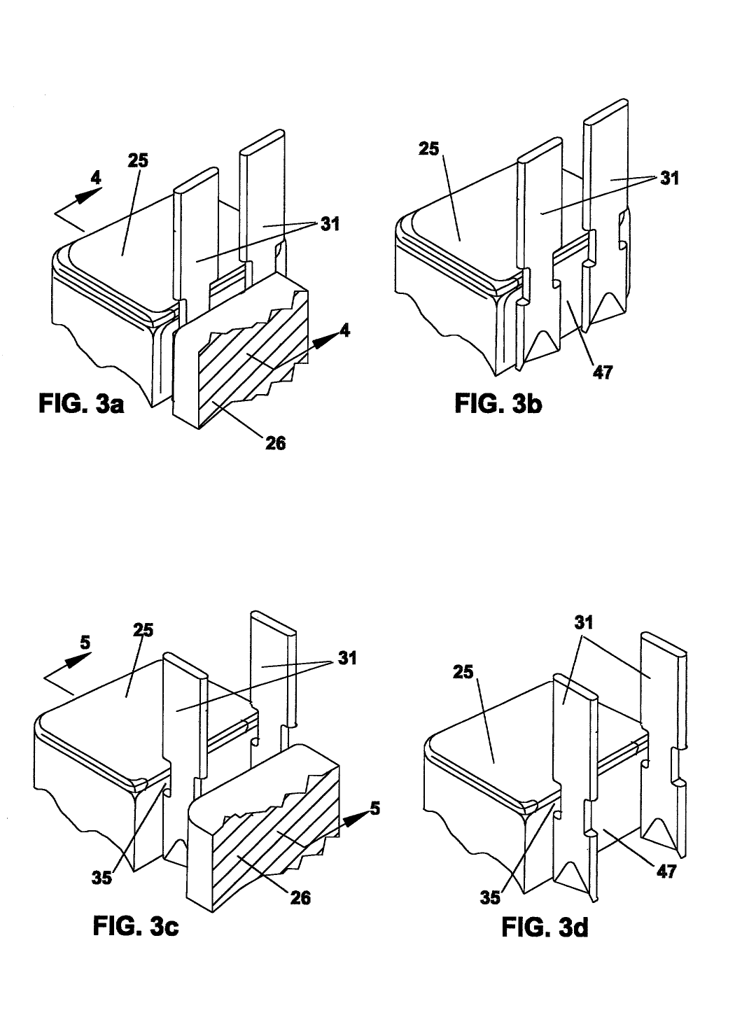

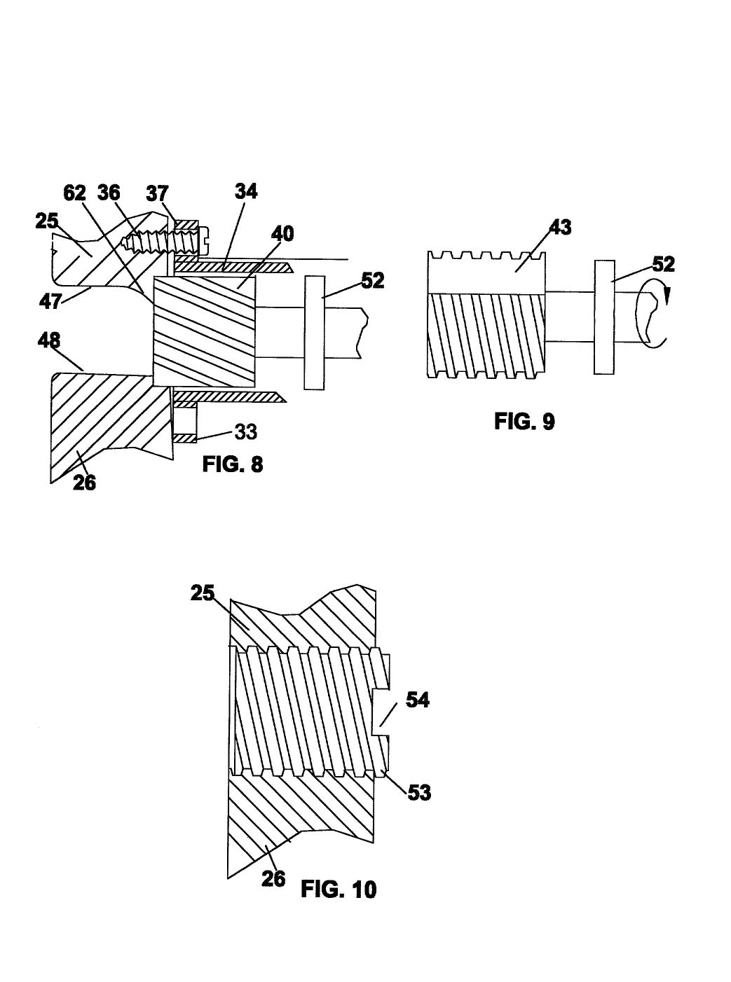

As shown in FIG. 6 and 7 the surgeon now has the full opening of the flange to gain access to the distracted disc space to inspect the disc space and to remove disc or bone debris. No other system gives the surgeon this open access to the disc space. The tube threads 39 may be attached to the threads 38 in flange 33, to position tube 34 to ream, thread, and install the implant dowel. The surgeon may remove the tube before removing the flange. This will allow the surgeon to inspect the implant after the implantation. The implant may also be placed through the flange with the tube removed. An alternate flange 44 contains open slots 46 so the flange may be installed on the distractors from the side. This system, with flange 33 also allows a clearance notch 35 shown in FIG. 3c and 3d machined into the flange to clear the front of the vertebral protrusion 62 (FIG. 6, this will allow the flange to be clamped to the vertebra body 25 rather than against the protrusion. This clamping will add more rigidity and better alignment of the flange and ultimately the guide tube 34.

The Guide Tube

Once the flange 33 is fixed with the distractor or the attachment screws 36, tube 34 is threaded into the flange to guide the tools. The tube external threads 39 are threaded into the flange internal threads 38. The tube inside diameter is sized to accommodate the tool-centering collar 52 with ample clearance. The tube assures that the reamer 40 and the tap 43 will be centered on the vertebral end plates 47 and 48 and parallel to the end plates to facilitate removing an appropriate amount of plate and bone from each adjacent vertebra. The machining tools and implant may be inserted through the tube 34. The tube may be threaded into the flange or it may be attached by other means.

The Vertebrae Machining Tools

The tube 34 assures that the tools 40 and 43 in FIGS. 8 and 9 will be centered on and parallel to the vertebral end plates 47 and 48. A reamer 40 is used to machine the fusing surfaces to prepare for threading the vertebral end plates 47 and 48. The tool guidance tube 34 facilitates removing an appropriate amount from each adjacent vertebra 25 and 26. After the vertebral end plate surfaces are machined to the proper size and bone exposure, a threading tap 43, which will accommodate the selected implant, will cut the internal thread. The machining tools will pass through the matching tube. The reaming, tapping, and implant insertion operations are well known to those skilled in the art. The tube may be removed to allow direct visualization during implant placement.

The Implant

After the machining is completed, an implant, preferably a threaded bone dowel 53, is screwed into the threaded hole. It is installed with a purpose built screwdriver or other installation means inserted into slot 54. The implant may also be fabricated differently to accommodate other driving or inserting means. The implant may have other features, which will hold it to the driving device as it is being manipulated into position for insertion. The preferred embodiment shows a bone screw 53 in FIG. 10 to be implanted for bone fusion across an intervertebral space, following the removal of a damaged disc. Such implants are structurally load-bearing devices, capable of withstanding the forces supported by the upper vertebrae 25. The dowel must be inserted before the distraction stabilizer is removed. The implant of the present invention has a thread to maintain the position. The implant 53 must be in contact with the circular arcs machined in the vertebral end plates 47 and 48. The implant may be fabricated from metal, nonmetals, polymers, biodegradable materials, bioabsorbable materials, allograft or autograft materials. Several sizes of dowel implants will be available for the surgeon to select from it at the time of surgery.

The Method

The implantation method allows several variations that will insure a higher rate of fusion success. These include the opportunity to provide more complete implant site inspections and versatility. Before the procedure is started the surgeon estimates the size of the dowel required. If necessary, some of the disc or vertebral debris may be removed. The procedure is conducted as follows: The distractors 31 are inserted between the vertebrae 25 and 26 into the collapsed disc space (FIG. 3a). The distractors 31 are rotated 90 degrees to spread the disc space to restore the intervertebral height and obtain adequate ligamentotaxis (FIG. 3c) . The guide flange slots 32 are placed over or alongside the distractor, as shown in FIG. 2, and moved into place with the distractors in the slots to insure that the flange will be centered on the vertebrae and in proper orientation to the end plates.

While centered over the distractor, the flange 33 is fixed to the two vertebrae 25 and 26 with the attachment screws 36 placed through the holes 37. The attachment screws may be threaded into the adjacent vertebra to maintain the vertebrae relative position while using the tooling 40 and 43. The distractors may be left in place and held by the flange clamps 45 to eliminate the need for flange-to-bone attachment screws. At the surgeon's option, a starting hole may be drilled in the vertebra, prior to threading the flange attachment screws 36 into the vertebra 25 and 26. (FIGS. 5 and 6). Also the screws maybe self-tapping.

The surgeon may inspect the vertebral end plates 47 and 48 and the intervertebral disc space. The tube 34 is threaded into place on the flange to guide the tools. (FIG. 1 and 2) The reamer 40 is inserted into the tube 34 and rotated to cut cylindrical arcs into the vertebrae (FIG. 8). The tube may be removed to determine if sufficient bone and disc is removed. After the hole is reamed, the tap 43 is inserted in the tube and is rotated by hand to thread the arcs in the vertebral end plates (FIG. 9). The tube is removed from the flange 33 and the threads are inspected (FIG. 10).

If the threads are acceptable, the prepared dowel 53 is threaded into the disc space. With the tube removed, the surgeon can measure the depth of the hole and select a dowel of the correct length and can visually insert it to the desired depth. It can be inserted nearer to the posterior vertebral body margin to decrease the lordosis or the anterior margin to increase the lordosis. Once the dowel position is acceptable, the flange 33 is removed to release the distraction held by the flange, allowing the dowel 53 to support the vertebral force. These machining and dowel insertion operations are well known to those skilled in the art.

We claim:

1. A device and a method for distracting and exposing vertebrae in a human spine in preparation for; machining, tooling, joining, stabilizing or repositioning of one or more vertebrae with respect to another vertebra, disc space, implant, or other bone/disc replacement material comprising:
(a) one or more cam action spreaders, serving to distract and/or align the end plates,
(b) a flange which attaches to the vertebrae or is held by the said spreaders which may be guided and/or supported by the cam action spreaders,
(c) attachment(s) which connect to the flange to guide tooling and/or implant placement of the vertebrae.

2. The method of claim 1 wherein a portion of the device is placed between two adjacent vertebrae, sections, of the spine, or into a disc joint.

3. The method of claim 1, wherein said spreaders may be left in position while the implantation takes place.

4. The method of claim 1, wherein the cam spreader is rotated around its axis to distract a part of the vertebral column.

5. The device of claim 1, wherein the spreader is flat, curved, paddle shaped or of other geometry to accommodate spinal geometry, in preparation for vertebral machining and/or tooling for implantation.

6. The device of claim 1, wherein the said spreader has sharp edges or ends to facilitate passage of a portion of the device into the spine.

7. The method of claim 1, where the device is placed before, during or after machining/tooling of the vertebral bone or disc.

8. The device of claim 1, where said device is made of metal.

9. The method of claim 1, wherein the spine is prepared for subsequent clamping to maintain contact and compression with said implant or graft or other vertebrae.

10. The device of claim 1, which provides for maintained distraction by configuration of the device, with a spring, metal, non-metal, an elastic material, or other force or displacement generating means.

11. The device of claim 1, wherein said device is fabricated by using mechanical attachments to fix bars, rods, tubes, or other devices for the purpose of machining/tooling or measuring the spine.

12. The device and method of claim 1 whereas a means is provided to adjust the inter- or intra-vertebral height and/or the vertebral angle(s).

13. The device and method of claim 1 in which soft tissue retraction is obtained or facilitated.

14. A device for distracting vertebrae in a human spine in preparation for, machining, tooling, joining, stabilizing or repositioning one or more vertebrae with respect to another vertebra, a disc space, an implant, or other bone/disc replacement material comprising:
(a) one or more cam action spreaders, serving to distract and/or align the vertebral end plates,
(b) a flange which attaches to the vertebrae or is held by the said spreaders which may be guided and/ or supported by the cam action spreaders.
(c) an attachment which connects to the flange to guide tooling of the vertebrae.

15. The device of claim 14 wherein the flange is attached to the vertebrae with screws, pins, bonding agent or other bone attachment means.

16. The device of claim 14 wherein the flange is composed of metal, non-metals, polymer, composite of materials, or combination of different materials

17. The device of claim 14 wherein the flange incorporates extensions to be used to guide tooling/machining of the vertebrae.

18. The device of claim 14 wherein the flange extensions consist of bars, rods, tubes, or other devices for the purpose of machining/tooling or measuring the spine

19. The device of claim 14 wherein flange extensions consist of bars, rods, tubes of varying geometry to accommodate a variety of existing or to be developed vertebral machining/tooling instruments/devices.

20. The device of claim 14 wherein the said device is made from one or more parts.

21. The device of claim 14 wherein the spine or section thereof may be held in distraction to varying degrees.

22. The device of claim 14 where the vertebral alignment may be manipulated or altered.

23. The device of claim 14 where other devices may be used as adjuncts to distract, manipulate, machine/tool or implant the spine.

24. The device of claim 14 which will allow placement of spinal implants including biological material(s), absorbable material(s), resorbable material(s), bone or other allograft, xenograft or autograft material(s), metals, non metals, plastics, polymers or materials to be developed for spinal implantation.

25. The device of claim 14 whereas distraction is maintained with a spring, an elastic material, or other force or displacement generating means.

26. The device of claim 14 whereas grooves or other modifications to the said spreaders or flange sections allow or assist in distraction or alteration of spinal alignment.

27. A method for distracting vertebrae in a human spine in preparation for; machining, tooling, joining, stabilizing or repositioning one or more vertebrae with respect to another vertebra, a disc space, an implant, or other bone/disc replacement material using:
(a) a means to maintain vertebral position and/or alignment,
(b) a means of attachment to the spine using screws, pins, bonding or other attachment means.
(c) an implant which can be left to maintain the alignment after surgical closure.

28. The method of claim 27 wherein a portion of the device is placed onto one or more vertebrae.

29. The method of claim 27, wherein a portion of the device is placed onto or between two adjacent sections of the spine or into a disc joint.

30. The method of claim 27, where the spreading and/or alignment is affected before, during or after machining/tooling of the, vertebral bone or disc.

31. The method of claim 27, providing accurate distraction of two adjacent sections of the spine.

32. The method of claim 27, wherein the spine is prepared for subsequent clamping to maintain contact and compression with the implant or other vertebrae.

33. The method of claim 27, whereas a means is provided to adjust the inter- or intra-vertebral height and/or the vertebral angle(s).

34. The method of claim 27 wherein non-spinal soft tissues are retracted to facilitate spinal exposure.
